SEQUENCE LISTING (4 sequences)

SEQ ID NO: 1 - DNA, 3851 bp, Ralstonia eutropha H16
  CDS (1)..(1767): phbC
atggcgaccggcaaaggcgcggcagcttccacgcaggaaggcaagtcccaaccattcaaggtcacgccggggccattcgatccagccacatggctggaatggtcccgccagtggcagggcactgaaggcaacggccacgcggccgcgtccggcattccgggcctggatgcgctggcaggcgtcaagatcgcgccggcgcagctgggtgatatccagcagcgctacatgaaggacttctcagcgctgtggcaggccatggccgagggcaaggccgaggccaccggtccgctgcacgaccggcgcttcgccggcgacgcatggcgcaccaacctcccatatcgcttcgctgccgcgttctacctgctcaatgcgcgcgccttgaccgagctggccgatgccgtcgaggccgatgccaagacccgccagcgcatccgcttcgcgatctcgcaatgggtcgatgcgatgtcgcccgccaacttccttgccaccaatcccgaggcgcagcgcctgctgatcgagtcgggcggcgaatcgctgcgtgccggcgtgcgcaacatgatggaagacctgacacgcggcaagatctcgcagaccgacgagagcgcgtttgaggtcggccgcaatgtcgcggtgaccgaaggcgccgtggtcttcgagaacgagtacttccagctgttgcagtacaagccgctgaccgacaaggtgcacgcgcgcccgctgctgatggtgccgccgtgcatcaacaagtactacatcctggacctgcagccggagagctcgctggtgcgccatgtggtggagcagggacatacggtgtttctggtgtcgtggcgcaatccggacgccagcatggccggcagcacctgggacgactacatcgagcacgcggccatccgcgccatcgaagtcgcgcgcgacatcagcggccaggacaagatcaacgtgctcggcttctgcgtgggcggcaccattgtctcgaccgcgctggcggtgctggccgcgcgcggcgagcacccggccgccagcgtcacgctgctgaccacgctgctggactttgccgacacgggcatcctcgacgtctttgtcgacgagggccatgtgcagttgcgcgaggccacgctgggcggcggcgccggcgcgccgtgcgcgctgctgcgcggccttgagctggccaataccttctcgttcttgcgcccgaacgacctggtgtggaactacgtggtcgacaactacctgaagggcaacacgccggtgccgttcgacctgctgttctggaacggcgacgccaccaacctgccggggccgtggtactgctggtacctgcgccacacctacctgcagaacgagctcaaggtaccgggcaagctgaccgtgtgcggcgtgccggtggacctggccagcatcgacgtgccgacctatatctacggctcgcgcgaagaccatatcgtgccgtggaccgcggcctatgcctcgaccgcgctgctggcgaacaagctgcgcttcgtgctgggtgcgtcgggccatatcgccggtgtgatcaacccgccggccaagaacaagcgcagccactggactaacgatgcgctgccggagtcgccgcagcaatggctggccggcgccatcgagcatcacggcagctggtggccggactggaccgcatggctggccgggcaggccggcgcgaaacgcgccgcgcccgccaactatggcaatgcgcgctatcgcgcaatcgaacccgcgcctgggcgatacgtcaaagccaaggcatgacgcttgcatgagtgccggcgtgcgtcatgcacggcgccggcaggcctgcaggttccctcccgtttccattgaaaggactacacaatgactgacgttgtcatcgtatccgccgcccgcaccgcggtcggcaagtttggcggctcgctggccaagatcccggcaccggaactgggtgccgtggtcatcaaggccgcgctggagcgcgccggcgtcaagccggagcaggtgagcgaagtcatcatgggccaggtgctgaccgccggttcgggccagaaccccgcacgccaggccgcgatcaaggccggcctgccggcgatggtgccggccatgaccatcaacaaggtgtgcggctcgggcctgaaggccgtgatgctggccgccaacgcgatcatggcgggcgacgccgagatcgtggtggccggcggccaggaaaacatgagcgccgccccgcacgtgctgccgggctcgcgcgatggtttccgcatgggcgatgccaagctggtcgacaccatgatcgtcgacggcctgtgggacgtgtacaaccagtaccacatgggcatcaccgccgagaacgtggccaaggaatacggcatcacacgcgaggcgcaggatgagttcgccgtcggctcgcagaacaaggccgaagccgcgcagaaggccggcaagtttgacgaagagatcgtcccggtgctgatcccgcagcgcaagggcgacccggtggccttcaagaccgacgagttcgtgcgccagggcgccacgctggacagcatgtccggcctcaagcccgccttcgacaaggccggcacggtgaccgcggccaacgcctcgggcctgaacgacggcgccgccgcggtggtggtgatgtcggcggccaaggccaaggaactgggcctgaccccgctggccacgatcaagagctatgccaacgccggtgtcgatcccaaggtgatgggcatgggcccggtgccggcctccaagcgcgccctgtcgcgcgccgagtggaccccgcaagacctggacctgatggagatcaacgaggcctttgccgcgcaggcgctggcggtgcaccagcagatgggctgggacacctccaaggtcaatgtgaacggcggcgccatcgccatcggccacccgatcggcgcgtcgggctgccgtatcctggtgacgctgctgcacgagatgaagcgccgtgacgcgaagaagggcctggcctcgctgtgcatcggcggcggcatgggcgtggcgctggcagtcgagcgcaaataaggaaggggttttccggggccgcgcgcggttggcgcggacccggcgacgataacgaagccaatcaaggagtggacatgactcagcgcattgcgtatgtgaccggcggcatgggtggtatcggaaccgccatttgccagcggctggccaaggatggctttcgtgtggtggccggttgcggccccaactcgccgcgccgcgaaaagtggctggagcagcagaaggccctgggcttcgatttcattgcctcggaaggcaatgtggctgactgggactcgaccaagaccgcattcgacaaggtcaagtccgaggtcggcgaggttgatgtgctgatcaacaacgccggtatcacccgcgacgtggtgttccgcaagatgacccgcgccgactgggatgcggtgatcgacaccaacctgacctcgctgttcaacgtcaccaagcaggtgatcgacggcatggccgaccgtggctggggccgcatcgtcaacatctcgtcggtgaacgggcagaagggccagttcggccagaccaactactccaccgccaaggccggcctgcatggcttcaccatggcactggcgcaggaagtggcgaccaagggcgtgaccgtcaacacggtctctccgggctatatcgccaccgacatggtcaaggcgatccgccaggacgtgctcgacaagatcgtcgcgacgatcccggtcaagcgcctgggcctgccggaagagatcgcctcgatctgcgcctggttgtcgtcggaggagtccggtttctcgaccggcgccgacttctcgctcaacggcggcctgcatatgggctga

SEQ ID NO: 2 - protein, 589 aa, Ralstonia eutropha H16
MATGKGAAASTQEGKSQPFKVTPGPFDPATWLEWSRQWQGTEGNGHAAASGIPGLDALAGVKIAPAQLGDIQQRYMKDFSALWQAMAEGKAEATGPLHDRRFAGDAWRTNLPYRFAAAFYLLNARALTELADAVEADAKTRQRIRFAISQWVDAMSPANFLATNPEAQRLLIESGGESLRAGVRNMMEDLTRGKISQTDESAFEVGRNVAVTEGAVVFENEYFQLLQYKPLTDKVHARPLLMVPPCINKYYILDLQPESSLVRHVVEQGHTVFLVSWRNPDASMAGSTWDDYIEHAAIRAIEVARDISGQDKINVLGFCVGGTIVSTALAVLAARGEHPAASVTLLTTLLDFADTGILDVFVDEGHVQLREATLGGGAGAPCALLRGLELANTFSFLRPNDLVWNYVVDNYLKGNTPVPFDLLFWNGDATNLPGPWYCWYLRHTYLQNELKVPGKLTVCGVPVDLASIDVPTYIYGSREDHIVPWTAAYASTALLANKLRFVLGASGHIAGVINPPAKNKRSHWTNDALPESPQQWLAGAIEHHGSWWPDWTAWLAGQAGAKRAAPANYGNARYRAIEPAPGRYVKAKA

SEQ ID NO: 3 - protein, 393 aa, Ralstonia eutropha H16
MTDVVIVSAARTAVGKFGGSLAKIPAPELGAVVIKAALERAGVKPEQVSEVIMGQVLTAGSGQNPARQAAIKAGLPAMVPAMTINKVCGSGLKAVMLAANAIMAGDAEIVVAGGQENMSAAPHVLPGSRDGFRMGDAKLVDTMIVDGLWDVYNQYHMGITAENVAKEYGITREAQDEFAVGSQNKAEAAQKAGKFDEEIVPVLIPQRKGDPVAFKTDEFVRQGATLDSMSGLKPAFDKAGTVTAANASGLNDGAAAVVVMSAAKAKELGLTPLATIKSYANAGVDPKVMGMGPVPASKRALSRAEWTPQDLDLMEINEAFAAQALAVHQQMGWDTSKVNVNGGAIAIGHPIGASGCRILVTLLHEMKRRDAKKGLASLCIGGGMGVALAVERK

SEQ ID NO: 4 - protein, 246 aa, Ralstonia eutropha H16
MTQRIAYVTGGMGGIGTAICQRLAKDGFRVVAGCGPNSPRREKWLEQQKALGFDFIASEGNVADWDSTKTAFDKVKSEVGEVDVLINNAGITRDVVFRKMTRADWDAVIDTNLTSLFNVTKQVIDGMADRGWGRIVNISSVNGQKGQFGQTNYSTAKAGLHGFTMALAQEVATKGVTVNTVSPGYIATDMVKAIRQDVLDKIVATIPVKRLGLPEEIASICAWLSSEESGFSTGADFSLNGGLHMG

(19) United States
(12) Patent Application Publication
(10) Pub. No.: US 2007/0269872 A1
(43) Pub. Date: Nov. 22, 2007

(54) Recombinant coryneform bacterium and method for producing diodegradable polyester

(75) Inventors: Seiichi Taguchi, Sapporo-shi (JP); Sung-Jin Jo, Sapporo-shi (JP)

Correspondence Address: QUINN EMANUEL;KODA & ANDROLIA, 865 S. FIGUEROA STREET, 10TH FLOOR, LOS ANGELES, CA, 90017, US

(73) Assignee: AgriBioIndustry Inc.

(21) Appl. No.: 11/471,986

(22) Filed: Jun. 21, 2006

(30) Foreign Application Priority Data
May 19, 2006 (JP) 2006-140882

(51) Int. Cl. C12P 7/62; C12P 7/40; C12N 1/21

(52) U.S. Cl. 435/135; 435/252.3; 435/136

(57) ABSTRACT

A recombinant coryneform bacterium obtained by providing a coryneform bacterium, which is known as a safe substance originally having no hazardous endotoxin, includes membrane structures different from E. coli and can be cultured in high density, with a biodegradable polyester producing ability, and a method for efficiently producing a biodegradable polyester to be contacted with a living organism for use in the medical and food industries formulated thereby. Specifically, the recombinant coryneform bacterium is obtained by modifying the coryneform bacterium so as to have the biodegradable polyester producing ability by introducing a biodegradable polyester synthetic enzyme gene group and a cell surface protein gene promoter from the coryneform bacterium into the coryneform bacterium.

### BACKGROUND OF THE INVENTION

1. Field of the Invention

The present invention generally relates to a recombinant coryneform bacterium and a method for producing a biodegradable polyester formulated thereby, and more particularly to a recombinant coryneform bacterium having an ability to efficiently produce a safe biodegradable polyester including no hazardous endotoxin by introducing a biodegradable polyester synthetic enzyme gene group and a cell surface protein gene promoter from the coryneform bacterium into the coryneform bacterium and a method for producing a biodegradable polyester formulated thereby.

2. Description of the Related Art

Since synthetic plastics derived from fossil fuels like petroleum are unable to be degraded in natural environment, they accumulate semipermanently in the environment, resulting in various environmental problems. Under the circumstances, much academic attention has been focused on biodegradable plastics that are degraded by naturally-existing microorganisms (known as an eco-friendly polymeric material), and the development of such material is being encouraged to provide excellent properties toward practical use. In view of biocompatibility of the material, the biodegradable plastics are expected to become leading biomaterials in biological and medical fields.

Currently, the biodegradable plastics are synthesized by microorganisms or chemicals, or derived from natural products. In particular, the microorganism synthesis approach is increasingly in high demand because of its advantage of using renewable biomass such as glucose and plant oils, leading to efficient resource utilization.

It has been conventionally known that many types of microorganisms produce biodegradable polyesters through a plurality of synthetic pathways after incorporating a biomass into the cell and substance accumulation can be found in a microbial cell body. The resulting synthesized biodegradable polyesters are extracted from a microbial cell to be formed and treated in various manners for several practical uses. Then, environmental microorganisms degrade used polyesters to carbon dioxide and water, thereby converting them into a recyclable starting biomass material. In particular, much attention is being focused on poly-3-hydroxyalkanoate, a type of biodegradable polyester having thermoplastic and favorable biodegradable properties as well as synthetic plastics.

An intracellular metabolic process for synthesizing poly-3-hydroxyalkanoate in a microorganism will be described. With a starting biomass material, (R)-3-hydroxyacyl-CoA, a monomer of poly-3-hydroxyalkanoate is produced through monomer feeding metabolic pathways, and a poly-3-hydroxyalkanoate synthetic enzyme polymerizes the (R)-3-hydroxyacyl-CoA to synthesize poly-3-hydroxyalkanoate.

In addition to some alternative pathways, the monomer feeding pathways include three main pathways: a pathway for dimerizing a starting substance of acetyl-CoA, a transduction pathway by an intermediate generated in de novo fatty acid synthetic pathway and another transduction pathway by an intermediate generated in β-oxidation.

In fact, when the biodegradable plastics are produced with naturally-producing bacteria, plastic degrading system thereof can be activated, thereby causing limited productivity improvement by artificial means and sometimes unwanted copolymerized composition in polyester production. Thus, this approach cannot assuredly produce desired biodegradable plastics due to the above mentioned complex microbial metabolic pathways, and it provides limited types of biodegradable plastics synthesized and a limited range of synthetic methods. In addition, some synthetic pathway control methods may produce copolymers, rather than intended homopolymers, and the resulting copolymers could be non-uniform in desired molar ratio.

Under the circumstances, the use of recently developed DNA recombinant techniques, in which genes of a biodegradable plastic synthetic enzyme are isolated to make microorganisms recombinant, is increasingly expected to produce practical biodegradable plastics. In this technological approach, the properties of resulting biodegradable plastics are modified according to each intended purpose, by improving the substance production due to the increase in the activity of the biodegradable plastic synthetic enzyme. Also, this modification can be achieved by controlling copolymer composition in the biodegradable polyesters by converting the substrate specificity of the biodegradable plastic synthetic enzyme. In general, E. coli is a major host for producing biodegradable plastics using these recombinant microorganisms.

Meanwhile, coryneform bacterium is classified as Gram-positive bacterium having neither polyester producing ability nor endotoxin in itself, and this strain ensures safe production of amino acids contained in human foods. Notably, the coryneform bacterium is capable of being cultured in high density, with its culture density over 10 times as E. coli. In this bacterium, all DNA sequences are completely decoded. The use of current DNA recombinant techniques, characterized by the development of amino-acid synthesis by incorporating a plasmid vector into a host, encourages the production of new substances. In many food companies, the coryneform bacterium is a leading amino acid fermentation bacterium. Despite this technological advance, there have been no reports on the production of biodegradable polyesters using the coryneform bacterium.

Meanwhile, the production of biodegradable polyesters, using a recombinant microorganism with a host E. coli, can cause unknown hazardous substances to be incorporated into an end product. Specifically, in conventional biodegradable polyester production methods, the host is mainly Gram-negative bacterium having endotoxin, such as E. coli, Ralstonia eutropha like a knockdown strain of poly-3-hydroxyalkanoate synthetic enzyme gene (PHB⁻⁴), and genus Pseudomonas like a knockdown strain of poly-3-hydroxyalkanoate synthetic enzyme gene, leading to the incorporation of the endotoxin into the polyesters. This problem is a major obstacle to the use of the biofunctional materials in medical and health food industries.

In addition, generally used biodegradable polyester producing approaches with microorganisms must be improved so as to optimize the productivity in a single cell and culture in high density optimized recombinant microorganisms at single cell level.

### SUMMARY OF THE INVENTION

It is, therefore, one object of the present invention to provide a recombinant coryneform bacterium obtained by providing a coryneform bacterium, which is known as a safe substance originally having no hazardous endotoxin includes membrane structures different from E. coli and can be cultured in high density, with a biodegradable polyester producing ability, and a method for efficiently producing a biodegradable polyester to be contacted with a living organism for use in the medical and food industries using the recombinant coryneform bacterium.

To solve the aforementioned problems, this inventor has focused on his own study, and successfully completed the present invention, in which a recombinant coryneform bacterium can be constructed so as to efficiently produce a biodegradable polyester capable of high-density culture by introducing and expressing a biodegradable polyester synthetic enzyme gene group with a coryneform bacterium as a host cell and a remarkably safe biodegradable polyester, containing no hazardous endotoxin, can be produced.

The recombinant coryneform bacterium according to the present invention is characterized by modifying a coryneform bacterium so as to have a biodegradable polyester producing ability by introducing a biodegradable polyester synthetic enzyme gene group and a cell surface protein gene promoter from said coryneform bacterium.

Preferably in this invention, the coryneform bacterium is provided with a transcriptional function by keeping closer the distance between said biodegradable polyester synthetic enzyme gene group and said cell surface protein gene promoter from said coryneform bacterium.

Also in this invention, said coryneform bacterium and said cell surface protein gene promoter from said coryneform bacterium are preferably genus Conynebacterium.

It is desirable that said genus Conynebacterium is Conynebacterium glutamicum.

Moreover, said Conynebacterium glutamicum is preferably Conynebacterium glutamicum ATCC13869.

In this invention, said biodegradable polyester synthetic enzyme gene group preferably includes a β-ketothiolase gene, an acetoacetyl-CoA reductase gene and a poly-3-hydroxyalkanoate synthetic enzyme gene.

Furthermore, it is desirable that said β-ketothiolase gene, said acetoacetyl-CoA reductase gene and said poly-3-hydroxyalkanoate synthetic enzyme gene form an operon.

Said biodegradable polyester synthetic enzyme gene group is preferably derived from genus Ralstonia.

The production of a biodegradable polyester according to the present invention is characterized by culturing said recombinant coryneform bacterium in a specified culture medium, containing glucose as a carbon source and ammonium sulfate as a nitrogen source in composition, at a culture temperature of about 27 to 37° C. and with a pH of about 7 to 8.

It is desirable that in this invention, said recombinant coryneform bacterium is cultured at a culture temperature of approximately 30° C. and with a pH of 7.5.

Preferably in this invention, the culture medium includes at least glucose, ammonium sulfate and biotin in composition, and the content of said glucose is over twice that of said ammonium sulfate.

Accordingly, it is, of course, that this invention can efficiently produce a biodegradable polyester that can be used as an eco-friendly, safe and highly functional material to be contacted with a living organism for use in the medical and food industries by obtaining a recombinant coryneform bacterium having a biodegradable polyester producing ability. This invention can also produce an amino acid and a biodegradable polyester independently in the switching mode in the same microbial cell body, thereby obtaining highly efficient microbial fermentation system, in which the total energy can be reduced in the production of the two types of high-value biological products.

### BRIEF DESCRIPTION OF THE DRAWINGS

The above and other objects of the invention will be seen by reference to the description taken in connection with the accompanying drawings, in which:

FIG. 1 is a chart showing a part of the metabolic pathway in the microbial cell body of the coryneform bacterium and the PHB synthetic pathway artificially constructed in the coryneform bacterium;

FIG. 2 is a table describing the culture medium composition for determining the optimal culture medium composition in the recombinant coryneform bacterium;

FIG. 3 is a graph showing the amount of polyesters synthesized according to a cultivation temperature in the recombinant coryneform bacterium;

FIG. 4 is a graph showing the amount of polyesters synthesized according to a culture pH in the recombinant coryneform bacterium;

FIG. 5 is a genetic construct for the three types of plasmid vectors constructed;

FIG. 6 is a graph showing the growth curve of the recombinant coryneform bacterium (indicated by black dots along the left vertical axis) and the total PHB synthesized in the cell (indicated by white dots along the right vertical axis), according to a cultivation time (along the horizontal axis) for the pPS-phbCAB-containing recombinant coryneform bacterium;

FIG. 7 is a chart showing the analysis of biodegradable polyesters produced by the pPS-phbCAB-containing recombinant coryneform bacterium using gas chromatography;

FIG. 8 is a table describing the molecular weights of PHB produced by the pPS-phbCAB-containing recombinant coryneform bacterium and E. coli;

FIG. 9A is a TEM photographic image showing PHB expression in the pPGEM-phbCAB-containing recombinant coryneform bacterium FIG. 9B is a TEM photographic image showing PHB expression in the pPS-phbCAB-containing recombinant coryneform bacterium.

### DETAILED DESCRIPTION OF THE INVENTION

A preferred embodiment of the recombinant coryneform bacterium and the method for producing biodegradable polyesters using the recombinant coryneform bacterium according to the present invention will be described.

The recombinant coryneform bacterium of this embodiment is a transformed coryneform bacterium provided with a biodegradable polyester producing ability by introducing a plasmid vector, containing a biodegradable polyester synthetic enzyme gene group (hereinafter enzyme gene group) and a cell surface protein B gene promoter from a coryneform bacterium originally having no biodegradable polyester producing ability, into said coryneform bacterium, using recombinant DNA techniques. The method for producing biodegradable polyesters using the recombinant coryneform bacterium of this embodiment can provide highly safe biodegradable polyesters by culturing said recombinant coryneform bacterium under specific conditions.

More specifically, the recombinant coryneform bacterium in this invention is obtained by providing the coryneform bacterium with a biodegradable polyester producing ability, by preparing a plasmid vector which bears genes containing the enzyme gene group and a promoter that can function in the coryneform bacterium coupled thereto, and introducing the plasmid vector into the coryneform bacterium to artificially construct a biodegradable polyester synthetic pathway in the bacterium.

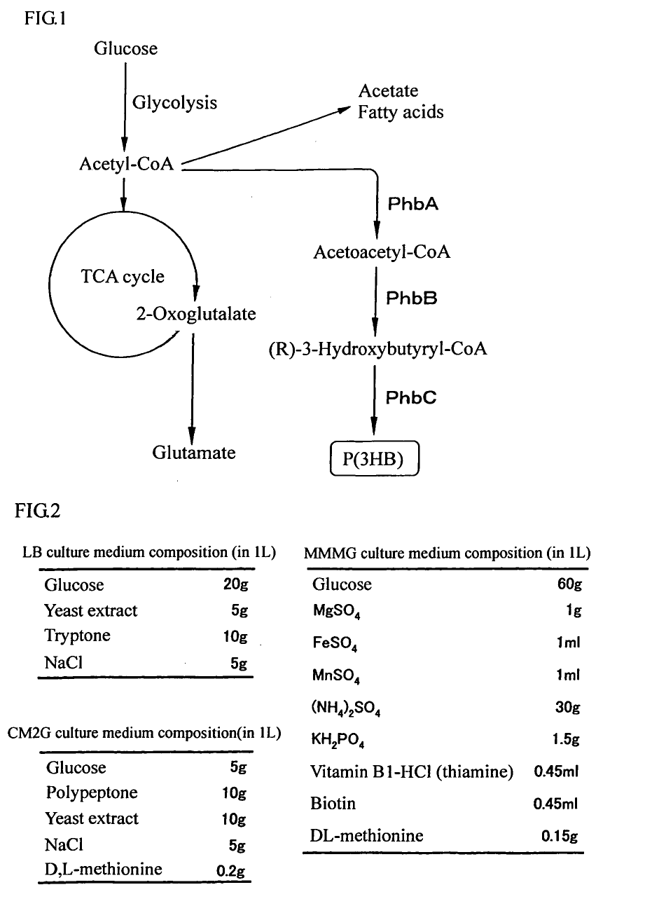

Here, a part of intracellular metabolic pathway in the coryneform bacterium and a biodegradable polyester synthetic pathway which is artificially constructed in the coryneform bacterium in this invention will be described. For instance, the construction of a pathway for synthesizing poly-3-hydroxybutyrate (PHB), a typical poly-3-hydroxyalkanoate (PHA) generated in the coryneform bacterium, will be described with reference to FIG. 1.

The coryneform bacterium first incorporates glucose into the cell and then glycolysis occurs to produce acetyl-CoA. Normally, this acetyl-CoA feeds into TCA cycle (a.k.a. tricarboxylic acid cycle or citric acid cycle), thereby synthesizing glutamic acid (amino acid) from 2-oxoglutaric acid (metabolic intermediate). In the coryneform bacterium, this amino acid synthetic route is already identified.

Meanwhile, it is suggested that already decoded DNA sequences in the coryneform bacterium show no existence of all 3 enzyme genes involved in PHB synthesis: β-ketothiolase (PhaA) gene, acetoacetyl-CoA reductase (PhaB) gene and PHB synthetic enzyme (PhaC) gene. Thus, it is believed that no PHB synthetic route is found in the coryneform bacterium and PHB synthesis is not actually confirmed.

Then, by introducing an operon of PHB synthetic enzyme gene group (hereinafter phaCAB) into the coryneform bacterium, a synthetic pathway, comprising a monomer feeding pathway by dimerizing acetyl-CoA, established by β-ketothiolase (PhaA) and acetoacetyl-CoA reductase (PhaB), and PHB synthetic enzyme (PhaC) directly coupled thereto, is designed. By this approach, PHB synthetic pathway is artificially constructed, using acetyl-CoA between the glycolysis and TCA cycle in the coryneform bacterium as a raw material.

In this invention, the promoter that can function in the coryneform bacterium is preferably a cell surface protein B gene promoter from the coryneform bacterium, which is joined to said enzyme gene group in a close range to provide a favorable transcriptional function.

More specifically, DNA sequences of said cell surface protein B gene promoter from the coryneform bacterium are required to be joined to a 5′ upstream region of the enzyme gene group to express said enzyme gene group in the coryneform bacterium. This joining position is not particularly limited if the enzyme gene group can be expressed, however, it is desirable that the enzyme gene group and the promoter contain no DNA sequences of a promoter derived from the same living organism as the enzyme gene group therebetween. Moreover, the connection is preferably made at small intervals, rather than at no intervals.

In addition, the coryneform bacterium of this embodiment is a bacterium with the following characteristics. First, it originally has no biodegradable polyester producing ability. Second, it is Gram-positive bacterium containing no endotoxin whose membrane structures are different from those of Gram-negative bacterium like E. coli. Third, it is a bacterium traditionally used as an amino acid fermentation bacterium, and its producing substances are strongly believed to be safe. In view of its cell culture density over ten times as E. coli, this type of bacterium is overall advantageous to the production of new substances. All of its DNA sequences are decoded and the development of highly usable host vector system is encouraged. Therefore, the use of these characteristics can efficiently synthesize significantly safe biodegradable polyesters.

In fact, Gram-positive bacteria which contain no endotoxin include e.g. Bacillus subtilis. However, this strain is sporulated as opposed to the coryneform bacterium, thereby providing insufficient properties for industrial use. In view of this disadvantage and the above-mentioned coryneform bacterium's characteristics, the coryneform bacterium is a Gram-positive bacterium suitable for industrial production.

A coryneform bacterium used as a host in this invention is not particularly limited, if it is provided with a biodegradable polyester producing ability having the above-mentioned characteristics. The coryneform bacterium is preferably bacterium that is classified as genus Agrococcus, genus Agromyces, genus Arthrobacter, genus Aureobacterium, genus Brevibacterium, genus Cellulomonas, genus Clavibacter, genus Microbacterium, genus Rathayibacter, genus Terrabacter, or genus Turicella, and more preferably genus Conynebacterium.

Moreover, the genus Conynebacterium is preferably a strain like Conynebacterium glutamicum ATCC13032, Conynebacterium glutamicum ATCC13032, Conynebacterium glutamicum ATCC13870, Corynebacterium and callunae ATCC15991, Corynebacterium and acetoglutamicum ATCC15806. In view of polyester producing ability, it is desirable that the genus Conynebacterium is Conynebacterium glutamicum ATCC13869.

A coryneform bacterium used as the cell surface protein B gene promoter from said coryneform bacterium in this invention is not particularly limited, if it is the same type of said coryneform bacterium used as a host. Preferably, in this invention, the coryneform bacterium is genus Conynebacterium like the aforementioned corynebacteria, and more specifically Conynebacterium glutamicum ATCC13869. The DNA sequences of the above promoter may include one or more base pair substitutions, deletions, or additions.

Also, the enzyme gene group in this invention is not particularly limited, if it can synthesize biodegradable polyesters from biomass such as sugars and plant oils. However, the enzyme gene group is preferably a PHA synthetic enzyme gene group that encodes bacteria-derived enzymes.

As for the enzyme gene group, the following 3 specific enzyme gene groups may be appropriate.
1. An enzyme gene group, containing β-ketothiolase, acetoacetyl-CoA reductase and PHB synthetic enzyme, that polymerizes PHB after an acetyl-CoA is converted into a monomer (R)-3-hydroxybutyryl-CoA over a pathway for the dimerizing the acetyl-CoA.
2. An enzyme gene group, containing (R)-specific enoyl-CoA hydrase and PHA synthetic enzyme, that polymerizes PHA after an enoyl-CoA (an intermediate of fatty acid-beta-oxidation system) is converted into a monomer (R)-3-hydroxyacyl-CoA.
3. An enzyme gene group, containing acyltransferase and PHA synthetic enzyme, that polymerizes PHA after (R)-3-hydroxyalkanoic acid-acyl carrier protein (an intermediate of de novo fatty acid synthesis system) is converted into (R)-3-hydroxyacyl-CoA.

In view of completely identified gene sequences, operon formation and easy-to-handle property, this embodiment employs the enzyme gene group as shown in the above item 1.

Here, PHB synthetic pathway over a pathway for dimerizing acetyl-CoA using acetyl-CoA will be described. First, two molecules of acetyl-CoA are condensed due to β-ketothiolase and converted into acetoacetyl-CoA. Subsequently, the acetoacetyl-CoA is converted into a monomer (R)-3-hydroxybutyryl-CoA, using acetoacetyl-CoA reductase with NADP reduction. PHB synthetic enzyme synthesizes PHB by the polymerization of (R)-3-hydroxybutyryl-CoA.

In host strains like Ralstonia eutropha, the above 3 biodegradable polyester synthetic enzyme gene groups form an operon phaCAB, bearing β-ketothiolase (PhaA) gene, acetoacetyl-CoA reductase (PhaB) gene and PHB synthetic enzyme (PhaC) gene, with various gene compositions determined by PHA-producing bacteria. Conversely, if said 3 biodegradable polyester synthetic enzyme gene groups form no such operon, it is desirable that by using state-of-the-art recombinant DNA techniques, an operon is artificially formed for use. This approach is aimed at minimizing plasmid for improving transformation efficiency and transcribing the 3 types of biodegradable polyester synthetic enzyme gene groups from one promoter in a synchronized and efficient manner.

In this invention, enzyme gene group-derived microorganisms include, but not particularly limited to, genus Ralstonia, genus Pseudomonas, genus Bacillus, genus Allochromatium, genus Synechocystis and genus Aeromonas, if they attain the objectives of this invention. Preferably, they include Ralstonia eutropha, and more specifically Ralstonia eutropha H16 strain. The Ralstonia eutropha means currently used Wautersia.

Next, a plasmid vector example that is introduced into the coryneform bacterium and constructed in this invention will be given in the following descriptions.

An enzyme gene group may be Ralstonia eutropha H16 strain-derived phbCAB. The base sequence of the enzyme gene group is shown in SEQ ID 1, and amino acid sequences that are encoded by the β-ketothiolase (PhaA) gene, acetoacetyl-CoA reductase (PhaB) gene and PHB synthetic enzyme (PhaC) gene in the gene group are shown in SEQ ID 2 to SEQ ID 4.

A host coryneform bacterium may be Conynebacterium glutamicum ATCC13869. Since the promoter is preferably the same as the host, the use of a cell surface protein B gene promoter of the Conynebacterium glutamicum ATCC13869 is desirable.

Then, the cell surface protein B gene promoter of the Conynebacterium glutamicum ATCC13869 is joined to a 5′ upstream region of said Ralstonia eutropha H16 strain-derived phbCAB.

A vector constructed is not particularly limited if it includes a plasmid that self-propagates in the coryneform bacterium, but it preferably self-propagates in E. coli as well. In this respect, the vector may be pPSPTG1 [Kikuchi, Y. et al.: Appl. Environmicrobiol, 69, 358-36 (2003)], a shuttle vector that can replicate both in the coryneform bacterium and E. coli.

By joining and inserting said enzyme gene group to the promoter-containing pPSPTG1 so as to join said promoter to a 5′ upstream region of said enzyme gene group of phbCAB, an expression plasmid vector of pGEM-phbCAB, that expresses phbCAB gene with said promoter, can be constructed.

From the above approaches, the plasmid vector can be constructed in the Conynebacterium glutamicum ATCC13869 to synthesize PHB.

The above constructed plasmid vector can be introduced into the coryneform bacterium according to several known methods, e.g. electroporation and calcium phosphate method. In these methods, a transformed recombinant Conynebacterium glutamicum ATCC13869, having a PHB producing ability, can be obtained.

The method for producing biodegradable polyesters in this invention is characterized by the extraction thereof from a culture obtained by culturing the above recombinant coryneform bacterium under specific culture conditions.

The specific culture conditions are not particularly limited if culture medium composition, cultivation temperature and pH conditions can achieve the growth of the coryneform bacterium and the production of biodegradable polyesters. Meanwhile, the following culture conditions are preferable, if the enzyme gene group is Ralstonia eutropha H16 strain-derived, the coryneform bacterium serving as a host and cell surface protein B gene promoter is Conynebacterium glutamicum ATCC13869, and the promoter is joined to an upstream region of the enzyme gene group to be provided with a transcriptional function.

In the culture medium, glucose and ammonium sulfate can be used as a carbon source and nitrogen source, respectively. More specifically, the culture medium includes at least glucose, ammonium sulfate and biotin, and the content of said glucose is preferably over twice that of said ammonium sulfate.

On the other hand, it is desirable that the cultivation temperature is about 27 to 37° C., more preferably 30° C. The culture pH is preferably in the range of about 7 to 8, more preferably 7.5.

The methods for recovering biodegradable polyesters from the coryneform bacterium include, but not particularly limited to, known solvent extraction, physical disintegration and chemical treatment methods. For example, after the biodegradable polyesters solve in organic solvents like chloroform, they can be extracted and purified by means of a specific reprecipitaion method using ethanol.

The biodegradable polyesters synthesized in the cell can be quantitated in the following method. Dry microbial cell body is converted into crotonic acid using concentrated sulfuric acid (elimination reaction) and 10 volumes of 0.014N sulfuric acid is added thereto. Then, using high performance liquid chromatography (HPLC), the biodegradable polyesters in a sample solution are separated from other components and the absorbance at 210 nm is spectroscopically detected. [Karr, D. B. et al: Appl. Environ Microbiol., 46, 1339-1344 (1983)]

The activity of PHB synthetic enzyme in the cell can be measured by quantitating CoA at a wavelength of 412 nm, that is released from a monomer substrate (R)-3-hydroxybutyryl-CoA during PHB polymerization reaction after the culture obtained is centrifuged to be recovered and its cells are disintegrated by supersonic treatment. [Satoh, Y., J. Biosci. Bioeng., 95, 335-341 (2003)]

Subsequently, after the biodegradable polyesters are extracted from the cell using organic solvents like chloroform, the composition thereof can be measured and analyzed by examining the extract in gas chromatography (GC) or nuclear magnetic resonance analysis (NMR).

The molecular weight of the biodegradable polyesters can be determined by means of gel permeation chromatography (GPC). The biodegradable polyester synthesis in the cell can be directly observed by transmission electron microscope (TEM).

Specific examples of this embodiment in this invention will be further described as follows.

### EXAMPLE 1

By introducing an operon of PHB synthetic gene group (phbCAB) by the electroporation [Libel, W. et al.: FEMS Microbiol. Lett., 65, 299-303 (1989)], culture medium composition, cultivation temperature and pH suitable for PHB synthesis were examined in Conynebacterium glutamicum ACTT13869 that expresses this gene group.

First, three types of culture media, LB culture medium, rich culture medium MCM2G [Kikuchi, Y. et al.: Appl. Environ. Microbial. 69, 358-36 (2003)] and minimal culture medium MMTG [Kikuchi, Y. et al.: Appl. Environ Microbiol., 69, 358-36 (2003)] were evaluated in composition at 30° C. for 72 hours after the cultivation. FIG. 2 shows the culture medium compositions for the above culture media.

It was found that only the minimal culture medium MMTG, including glucose as a carbon source and ammonium sulfate as a nitrogen source, achieved PHB synthesis. LB culture medium and MCM2G culture medium, mainly composed of natural culture medium, showed no PHB synthesis. From this observation, MMTG culture medium seems a favorable choice due to its completely identified composition and variable nutrient balance of carbon and nitrogen (known as C/N ratio) for synthesizing biodegradable polyesters using coryneform bacterium.

Next, the cultivation temperature and pH were discussed in an MMTG culture medium containing 50 μg/mL kanamycin. The temperature was in the range of 27 to 37° C., and the pH ranged from 7 to 8.

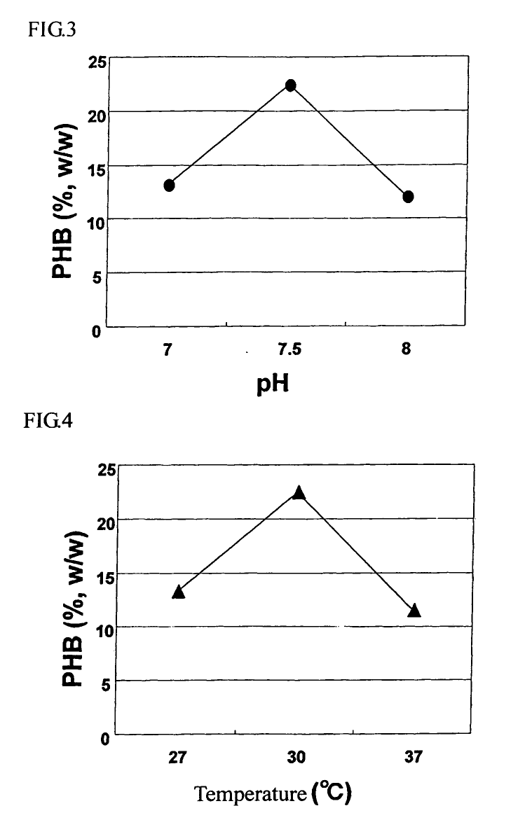

As shown in FIGS. 3 and 4, PHB synthesis was observed under any cultivation temperature and pH conditions, but the optimal synthesis temperature and pH were 37° C. and 7.5, respectively. These conditions corresponded to optimal culture conditions for coryneform bacterium propagation. It is suggested that in the synthesis of biodegradable polyesters in the coryneform bacterium, it is important to set culture conditions so as to be associated with coryneform bacterium propagation.

### EXAMPLE 2

To express the enzyme gene group of phbCAB in the coryneform bacterium, three types of plasmid vectors with different promoter patterns, containing a promoter in a 5′ upstream region and a terminator in a 3′ downstream region, are constructed in the following processes. FIG. 5 shows genetic constructs for the 3 types of plasmid vectors constructed.

The 3 promoter patterns were Ralstonia eutropha H16 strain-derived phbCAB promoter (Pphb), Pphb and a cell surface protein B gene promoter from the coryneform bacterium (Pcsp) combined, and Pcsp.

The coryneform bacterium was Conynebacterium glutamicum ATCC13869. The phbCAB was Ralstonia eutropha H16 strain-derived. The terminator was Ralstonia eutropha H16 strain-derived phbCAB terminator (Tphb). The plasmid was a shuttle vector pPSPTG1 that can replicate both in the coryneform bacterium and E. coli.

After pPSPTG1 plasmid was digested with a restriction enzyme of KpnI and it was made blunt with T4 DNA polymerase, a vector digested with a restriction enzyme of BamHI was obtained by gel extraction method. Then, gene fragments of about 5.0 kb, containing the Pphb promoter, the phbCAB enzyme gene group and a Tphb terminator, obtained by digesting pGEM-phbCAB plasmid [Taguchi, S. et al, FEMS Microbilolett., 198, 65-71 (2001)] with restriction enzymes of SmaI and BamHI, were inserted and joined to this vector. By this treatment, pPGEM-phbCAB expression plasmid vector, that expresses phbCAB gene with the Pphb promoter, was constructed (see FIG. 5 (A)).

After pPSPTG1 plasmid was digested with a restriction enzyme of BstEII and it was made blunt with T4 DNA polymerase, a vector digested with a restriction enzyme of BamHI was obtained by gel extraction method. Then, gene fragments of about 5.0 kb, containing the Pphb promoter, the phbCAB enzyme gene group and a Tphb terminator, obtained by digesting pGEM-phbCAB plasmid with restriction enzymes of SmaI and BamHI, were inserted and joined to this vector. By this treatment, pPSGEM-phbCAB expression plasmid vector, that expresses phbCAB gene with the Pphb and Pcsp promoters, was constructed (see FIG. 5 (B)).

After pPSPTG1 plasmid and pGEM-phbCAB plasmid were digested with restriction enzymes of BstEII and Csp45I, respectively and they were made blunt with T4 DNA polymerase, they were digested with a restriction enzyme of BamHI. Gene fragments of 4.3 kb, containing the phbCAB enzyme gene group and a Tphb terminator, obtained by using the gel extraction method, were ligated to the vector. By this treatment, pPS-phbCAB expression plasmid vector, that expresses phbCAB gene with the Pcsp promoter, was constructed (see FIG. 5 (C)).

The three types of plasmid vectors: pPGEM-phbCAB, pPSGEM-phbCAB and pPS-phbCAB were each introduced into the coryneform bacterium by means of the electroporation to obtain a transformed recombinant coryneform bacterium.

After the three types of recombinant coryneform bacteria were cultured in the MMTG culture medium at 30° C. and with a pH of 7.5 for 72 hours, PHB content was measured.

PHB accumulated in the microbial cell body can be quantitated in the following method. Dry microbial cell body was converted into crotonic acid using concentrated sulfuric acid and 10 volumes of 0.014N sulfuric acid was added thereto. Then, using HPLC, the PHB in a sample solution was separated from other components and the absorbance at 210 nm was spectroscopically detected. [Karr, D. B. et al: Appl. Environmicrobiol, 46, 1339-1344 (1983)]

More specifically, after adding a TE buffer solution to the microbial cell body and suspending it, the microbial cell body was recovered with centrifugal force. The microbial cell body was frozen at −80° C. for 2 hours, and it was vacuum-dried for 2 days to measure its dry weight. After 1 mL of sulfuric acid was added to the dry microbial cell body and it was heated with a heating block at 120° C. for 40 minute to be converted into crotonic acid, it was quenched with ice. Then, 4 ml of 0.014N sulfuric acid solution was gradually added to the sample, and agitated and cooled. The sample obtained passed through PTFE membranes with a hole diameter of 0.45 μm (Advance Mfs. Inc, Tokyo) and crotonic acid was measured with an absorbance at 210 nm using HPLC. The column was Aminex HPX-87H ion column (7.8 mml. D.×300 mm; Bio-Rad Lab., California) used at 60° C. and the mobile phase was 0.014N sulfuric acid solution with a flow rate of 0.7 mL/min. The PHB accumulation rate was determined using an efficiency of 50% for converting pure poly-3-hydroxybutyrate into crotonic acid, based on the relation between the amount of crotonic acid and area (calibration curve) obtained with HPLC.

As a result, no PHB was produced in the recombinant coryneform bacteria containing plasmid vectors of pPGEM-phbCAB and pPSGEM-phbCAB, but only the recombinant coryneform bacterium bearing a plasmid vector of pPS-phbCAB observed PHB production.

Moreover, after measuring the activity of PHB synthetic enzyme (phbC) in phbCAB, only the recombinant coryneform bacterium containing the pPS-phbCAB plasmid showed such activity.

The activity of PHB synthetic enzyme in the total extracted microbial cell body was measured by quantitating CoA at a wavelength of 412 nm, that is released from a monomer substrate (R)-3-hydroxybutyryl-CoA when the substrate reacts with the PHB enzyme. [Satoh, Y., J. Biosci. Bioeng., 95, 335-341 (2003)]

More specifically, the above recombinant coryneform bacteria were cultured at 30° C. for 72 hours. Then, 14,000×g of the recombinant coryneform bacteria were centrifuged for 2 minutes and the microbial cell body was disintegrated with supersonic disintegrator 15 times with ice for 4 seconds. Subsequently, 14,000×g of the recombinant coryneform bacteria were centrifuged for 2 minutes to obtain the total extracted microbial cell body. Next, IM potassium phosphate buffer solution (pH7.0), containing 4.08 mM (R)-3-hydroxybutyryl-CoA, was preheated at 25° C. for 10 minute, and said total extracted microbial cell body was added to the solution to cause enzyme reaction. After the mixture was sampled in a small portion of 20 μL, 50 μL of 5% TCA was added to the sampling solution and agitated to stop enzyme reaction. 337.5 μL of 500 mM potassium phosphate buffer solution (pH7.5) and 5 μL of 10 mM DTNB solution were added to 62.5 μL of a supernatant obtained by centrifuging the sample at 4° C. at a speed of 15,000 rpm for 10 minutes, and it was left unattended at room temperature for over 2 minutes. Afterward, the resultant TNB anion was measured at an absorbance of 412 nm (molar extinction coefficient: 13600). The amount of enzyme that produces 1 μmol of TNB anion with 1-minute reaction was set at 1 Unit.

These observations found that gene expression of phbCAB of the recombinant coryneform bacteria can be achieved by the cell surface protein B gene promoter (Pcsp) from the coryneform bacterium, not by the phbCAB promoter (Pphb). In addition, the phbC activity was confirmed in the recombinant coryneform bacterium provided with a phbCAB producing ability, and from the observation of an operon formed in phbCAB, it is suggested that monomer feeding enzymes of phbA and phbB are also functionally expressed.

### EXAMPLE 3

In the above considerations of the promoters, only the recombinant coryneform bacterium containing pPS-phbCAB showed PHB synthesis. It is thus estimated that the cell surface protein B gene promoter (Pcsp) from the coryneform bacterium contributed to phbCAB expression in the coryneform bacterium. From these findings, the recombinant coryneform bacterium having a PHB synthetic ability was cultured in the optimal MMTG culture medium at 30° C. and with a pH of 7.5 to determine the appropriate cultivation time.

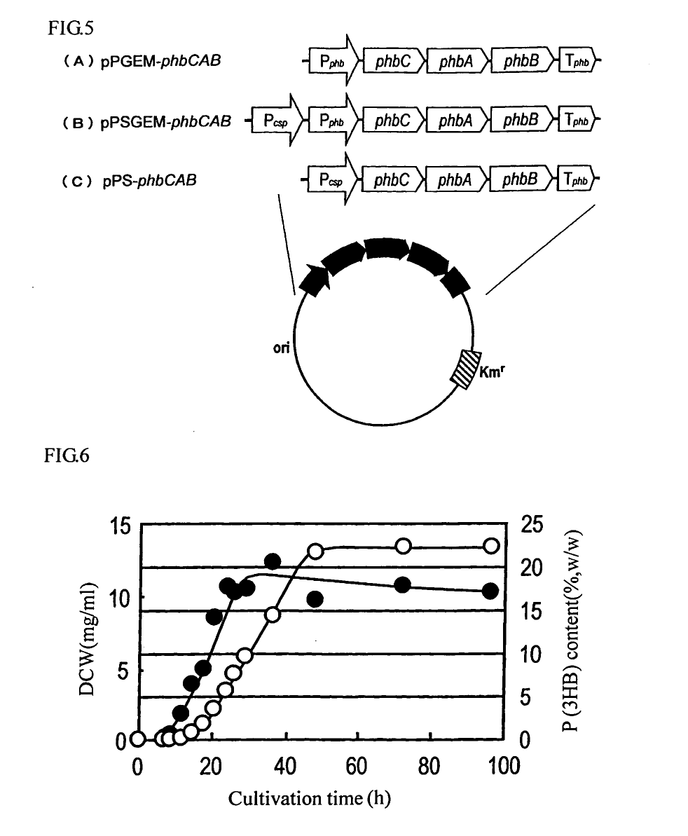

To find the optimal cultivation time, the growth of the recombinant coryneform bacterium and PHB synthesis were examined with time up to 96 hours. The growth curve for the recombinant coryneform bacterium was plotted by sampling the culture solution with time and measuring dry microbial cell body weight (in mg/mL) (indicated by black dots in FIG. 6). Meanwhile, the total PHB synthesized in the cell (in %, the ratio of PHB weight to dry microbial cell body weight) was plotted by sampling the culture solution simultaneously with the sampling for the growth curve and analyzing from the measured dry microbial cell body weight (indicated by white dots in FIG. 6).

Consequently, as shown in the FIG. 6, the recombinant coryneform bacterium's growth and PHB synthesis were closely associated with each other. In a more specific way, PHB synthesis attained a constant level of approx. 22.5% after 48 hours, and it demonstrated a stable accumulation up to 96 hours. From these observations, the cultivation time was set at 72 hours to determine the total PHB synthesized in the cell in a reproducible and stable manner.

### EXAMPLE 4

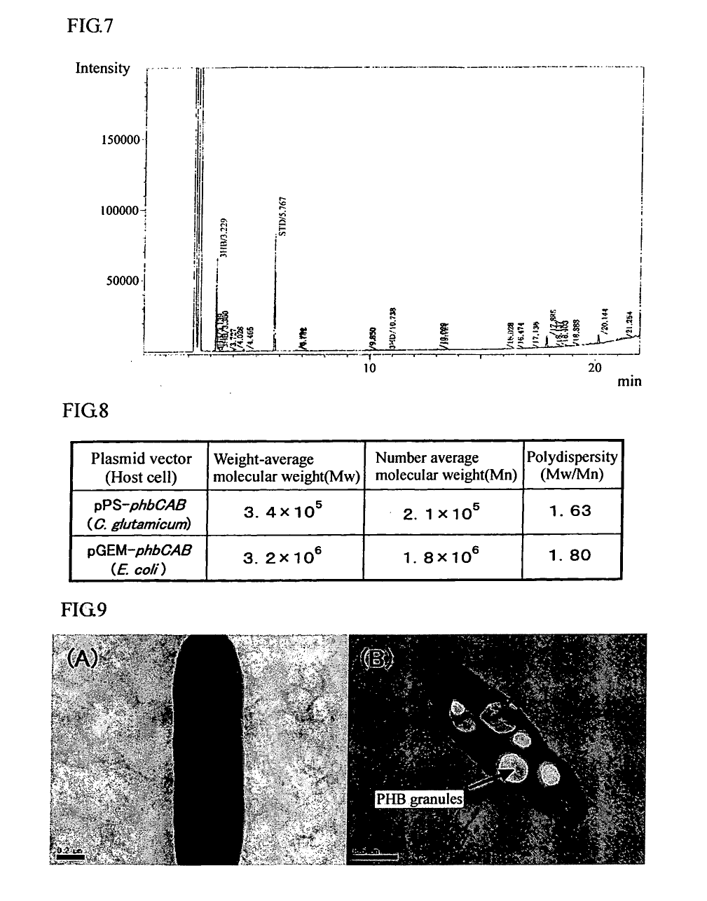

The composition of biodegradable polyesters produced by the pPS-phbCAB-containing recombinant coryneform bacterium was analyzed using gas chromatography (GC). As shown in FIG. 7, this analysis detected the peak corresponding only to methylesterform of 3HB. It was thus confirmed that biodegradable polyesters produced are homopolymers of (R)-3-hydroxybutyrate, not involving the introduction of monomer units of non-(R)-3-hydroxybutyrate.

### EXAMPLE 5

The molecular weight of PHB produced by the pPS-phbCAB-containing recombinant coryneform bacterium was measured using gel permeation chromatography (GPC).

The molecular weight was measured at 40° C., using Jasco GPC-900 equipped with TSK gel GMHHR-M column (7.8 mm I.D.×300 mm; Tosho Co., Tokyo) and Shodex XF-804Lcolumn (8 mm I.D.×300 mm; Showa Denko K. K., Tokyo). The mobile phase was chloroform with a flow rate of 0.8 mL/min. The calibration curve was determined using pure polystyrene.

Consequently, as shown in FIG. 8, the molecular weight was one-order smaller than that of PHB synthesized in a recombinant E. coli JM109 strain having a replaced host of E. coli.

### EXAMPLE 6

The expression of PHB in the recombinant coryneform bacteria containing pPGEM-phbCAB and pPS-phbCAB was observed using transmission electron microscope (TEM).

Specifically, the recombinant coryneform bacteria were treated in 2% glutaraldehyde mixed with cacogyl chloride buffer solution (pH7.4) for one hour and then in 2% osmium tetroxide for 30 minutes for double-immobilizing. The sample was dehydrated with ethanol substitution and it was embedded in an epoxy resin of Epon 812. After sections in epoxy resin were prepared and stained with uracil acetate and lead acetate, the recombinant coryneform bacteria were observed using electron microscope (JEM-2010; Jeol, Tokyo, Japan).

Consequently, as shown in FIG. 9, no granules were found in the pPGEM-phbCAB-containing recombinant coryneform bacterium (see FIG. 9 (A)), but only the pPS-phbCAB-containing recombinant coryneform bacterium clearly indicated a white mass of PHB granule (insoluble) (see FIG. 9 (B)).

According to the aforementioned embodiment, by providing a coryneform bacterium originally having no biodegradable polyester producing ability like wild-type coryneform bacterium with such an ability to obtain a recombinant coryneform bacterium, eco-friendly, highly safe and functional biodegradable polyesters to be contacted with a living organism for use in the medical and food industries can be efficiently produced. This invention can also produce amino acids and biodegradable polyesters in the same microbial cell body simultaneously, thereby obtaining highly efficient microbial fermentation system, in which the total energy can be reduced in the production of the two types of high-value biological products.

A recombinant coryneform bacterium having a biodegradable polyester producing ability of this embodiment and a method for producing a biodegradable polyester using the recombinant coryneform bacterium are not intended as a definition of the limits of the above description, but may be modified accordingly.

1. A recombinant coryneform bacterium obtained by modifying a coryneform bacterium so as to have a biodegradable polyester producing ability by introducing a biodegradable polyester synthetic enzyme gene group and a cell surface protein gene promoter from said coryneform bacterium into said coryneform bacterium.

2. The recombinant coryneform bacterium set forth in claim 1 wherein said coryneform bacterium is provided with a transcriptional function by keeping closer the distance between said biodegradable polyester synthetic enzyme group and said cell surface protein gene promoter from said coryneform bacterium.

3. The recombinant coryneform bacterium set forth in claim 1 wherein said coryneform bacterium and said cell surface protein gene promoter from said coryneform bacterium are genus Conynebacterium.

4. The recombinant coryneform bacterium set forth in claim 3 wherein said genus Conynebacterium is Conynebacterium glutamicum.

5. The recombinant coryneform bacterium set forth in claim 4 wherein said Conynebacterium glutamicum is Conynebacterium glutamicum ATCC13869.

6. The recombinant coryneform bacterium set forth in claim 1 wherein said biodegradable polyester synthetic enzyme gene group comprises a β-ketothiolase gene, an acetoacetyl-CoA reductase gene and a poly-3-hydroxyalkanoatesynthetic enzyme gene.

7. The recombinant coryneform bacterium set forth in claim 6 wherein said β-ketothiolase gene, said acetoacetyl-CoA reductase gene and said poly-3-hydroxyalkanoatesynthetic enzyme gene form an operon.

8. The recombinant coryneform bacterium set forth in claim 7 wherein said biodegradable polyester synthetic enzyme gene group is genus Ralstonia-derived.

9. A method for producing a biodegradable polyester by culturing said recombinant coryneform bacterium set forth in claim 1 in a specified culture medium, containing glucose as a carbon source and ammonium sulfate as a nitrogen source in composition at a culture temperature of about 27 to 37° C. and with a pH of about 7 to 8.

10. The method for producing a biodegradable polyester set forth in claim 9 wherein said recombinant coryneform bacterium is cultured at a cultivation temperature of about 30° C. and with a pH of about 7.5.

11. The method for producing a biodegradable polyester set forth in claim 9 wherein the culture medium includes at least glucose, ammonium sulfate and biotin in composition, and the content of said glucose is over twice that of said ammonium sulfate.
